(19) United States
(12) Patent Application Publication
(10) Pub. No.: US 2023/0037492 A1
(43) Pub. Date: Feb. 9, 2023

(54) VENTILATION SYSTEM WITH A SPEAKING FUNCTION

(71) Applicant: Loewenstein Medical Technology S.A., Luxembourg (LU)

(72) Inventors: Peter KREMEIER, Karlsruhe (DE); Christof GOEBEL, Hamburg (DE); Andreas SCHOBER, Frankfurt am Main (DE)

(21) Appl. No.: 17/817,822

(22) Filed: Aug. 5, 2022

(30) Foreign Application Priority Data
Aug. 7, 2021 (DE) 102021004079.1

(51) Int. Cl. A61M 16/04; A61M 16/00; A61M 16/20

(52) U.S. Cl. CPC A61M 16/0468; A61M 16/0003; A61M 16/202; A61M 2016/0027

(57) ABSTRACT

System for assisting a patient in speaking, comprising at least one ventilation apparatus and a patient interface, the ventilation apparatus comprising at least one controllable respiratory gas source and being designed to identify two or more respiratory phases, at least inspiration and expiration, of the patient, and the patient interface having at least one speaking tube and a respiratory tube and being configured to conduct speaking gas to the patient via the speaking tube and to conduct respiratory gas to and/or from the patient via the respiratory tube. The system is configured to provide speaking gas to the patient at least temporarily in a speaking mode in order to enable speaking.

### CROSS-REFERENCE TO RELATED APPLICATIONS

The present application claims priority under 35 U.S.C. § 119 of German Patent Application No. 102021004079.1, filed Aug. 7, 2021, the entire disclosure of which is expressly incorporated by reference herein.

### BACKGROUND OF THE INVENTION

### 1. Field of the Invention

The invention relates to a system for ventilation with a speaking function, and to a tracheal cannula which permits speaking during ventilation.

### 2. Discussion of Background Information

A frequent form of ventilation, in particular in the clinical sphere, is the ventilation of the patients via a tracheal cannula. To this end, use is made of a trachea section through which the cannula is introduced into the trachea, generally below the glottis, of the patient. In order very substantially to prevent slipping and penetration of secretion and pathogens, the cannulas are blocked via a balloon in the trachea. This also prevents respiratory gas from flowing through the glottis outside the tracheal cannula, and therefore speaking becomes impossible.

The prior art discloses tracheal cannulas which are not blocked and are controlled in such a manner that the patient can exhale outside the tracheal cannula. However, tracheal cannulas without a balloon (“cuff”) conceal the risk of excessive amounts of secretion and pathogens being able to pass into the patient's lungs. For some blocked tracheal cannulas, provision is made for them to be unblocked in order for the patient to speak, i.e. for the balloon to be emptied. However, this method is slow and is also unpleasant for the patient.

In view of the foregoing, it would be advantageous to have available a system which enables pleasant and naturally acting speaking of the patient and involves as little outlay on apparatus as possible.

### SUMMARY OF THE INVENTION

The present invention provides a system for assisting a patient in speaking, comprising at least one ventilation apparatus and a patient interface, the ventilation apparatus comprising at least one controllable respiratory gas source and being designed to identify the respiratory phases, but at least inspiration and expiration, of the patient, and the patient interface having at least one speaking tube and a respiratory tube and being configured to conduct speaking gas to the patient via the speaking tube and to conduct respiratory gas to and/or from the patient via the respiratory tube, wherein the system is configured to provide speaking gas to the patient at least temporarily in a speaking mode in order to enable speaking.

In some embodiments, it is a characteristic feature of the system that the patient interface is a tracheal cannula.

In some embodiments, it is a characteristic feature of the system that the system is configured to switch into the speaking mode only during the expiration of the patient.

In some embodiments, it is a characteristic feature of the system that the system is configured and designed to switch into a standby mode while the speaking mode is not active, wherein, in the standby mode, a lower pressure and/or smaller flow of speaking gas is present in the speaking tube than the pressure and/or flow in the speaking mode.

In some embodiments, it is a characteristic feature of the system that the system is configured and designed so as, during the standby mode, to predetermine a pressure and/or flow of speaking gas, at which the patient can close the glottis or can keep same closed.

In some embodiments, it is a characteristic feature of the system that the system is configured such that a speaking function can be activated and deactivated, with switching into the speaking mode being made possible only when the speaking function is activated.

In some embodiments, it is a characteristic feature of the system that the system is configured such that, when the speaking function is deactivated, no speaking gas is delivered to the speaking tube.

In some embodiments, it is a characteristic feature of the system that the system is configured to switch over periodically between the standby mode and the speaking mode, the system switching into the standby mode during the inspiration of the patient and switching into the speaking mode during the expiration of the patient.

In some embodiments, it is a characteristic feature of the system that the system is configured and designed to identify an intention of the patient to speak and switches into the speaking mode only when an intention of the patient to speak is identified.

In some embodiments, it is a characteristic feature of the system that the system is configured and designed to identify the intention of the patient to speak on the basis of a pressure drop and/or increase in flow of the speaking gas in the speaking tube.

In some embodiments, it is a characteristic feature of the system that the system is configured and designed to identify the intention of the patient to speak via at least one acceleration sensor and/or at least one microphone.

In some embodiments, it is a characteristic feature of the system that the ventilation apparatus is configured and designed to deliver speaking gas and respiratory gas.

In some embodiments, it is a characteristic of the system that the respiratory gas source is configured to deliver the speaking gas and the respiratory gas.

In some embodiments, it is a characteristic feature of the system that the system comprises a control valve which is connected in a gas-conducting manner at least to the speaking tube of the patient interface.

In some embodiments, it is a characteristic feature of the system that the control valve is connected to a Y piece, wherein the Y piece is connected at least to the respiratory tube of the patient interface and both respiratory gas and speaking gas are conducted through the control valve.

In some embodiments, it is a characteristic feature of the system that the system comprises a speaking controller, wherein the speaking controller is configured to control the control valve.

In some embodiments, it is a characteristic feature of the system that the system is configured to switch the valve in such a manner that, at least in the speaking mode, speaking gas is delivered to the speaking tube.

In some embodiments, it is a characteristic feature of the system that the valve is switchable in such a manner that, during the standby mode, a pressure and/or flow of speaking gas is maintained in the speaking tube, the pressure and/or flow being lower/smaller than the pressure and/or flow of the speaking gas in the speaking mode.

In some embodiments, it is a characteristic feature of the system that the valve and the speaking controller are integrated in the ventilation apparatus.

In some embodiments, it is a characteristic feature of the system that, in addition to the ventilation apparatus, the system comprises at least one speaking apparatus, wherein the speaking apparatus comprises at least one gas source which is configured to at least temporarily deliver speaking gas.

In some embodiments, it is a characteristic feature of the system that the speaking apparatus is configured to switch over between the speaking mode and the standby mode.

In some embodiments, it is a characteristic feature of the system that the speaking apparatus is connected to the ventilation apparatus.

In some embodiments, it is a characteristic feature of the system that the speaking apparatus is configured to receive information regarding the respiratory phases of the patient from the ventilation apparatus and to control the speaking gas flow on the basis of said information.

In some embodiments, it is a characteristic feature of the system that the ventilation apparatus is configured to control the speaking apparatus.

In some embodiments, it is a characteristic feature of the system that the speaking apparatus can be coupled to the ventilation apparatus.

In some embodiments, it is a characteristic feature of the system that the speaking apparatus is configured to identify the respiratory phases of the patient, at least the inspiration and expiration, and to switch into the speaking mode or the standby mode on the basis of the respiratory phases.

In some embodiments, it is a characteristic feature of the system that the speaking apparatus is configured and designed to identify an intention of the patient to speak and, when an intention to speak is identified, to switch into the speaking mode.

In some embodiments, it is a characteristic feature of the system that the speaking apparatus is designed as a ventilation apparatus.

In some embodiments, it is a characteristic feature of the system that the speaking apparatus comprises a fan which is configured for delivering speaking gas, and the speaking apparatus comprises a control unit which is configured and designed to control fans.

In some embodiments, it is a characteristic feature of the system that the speaking apparatus comprises a valve controller which is connected to the valve and is configured to switch the valve.

The invention further provides a method for controlling a delivery of speaking gas, wherein the speaking gas flow and/or speaking gas pressure in a speaking mode is predetermined to a first value and, in a standby mode, is set to a second value, the first value being higher than the second value and the first value being set in such a manner that it is made possible for a user to speak.

In some embodiments, it is a characteristic of the method that the switch is periodically made between the speaking mode and the standby mode.

In some embodiments, it is a characteristic feature of the method that switching over from the standby mode into the speaking mode presupposes that an intention to speak has been identified, wherein the intention to speak is identified by a drop in the speaking gas pressure during the standby mode and/or an increase in the speaking gas flow during the standby mode.

In some embodiments, it is a characteristic feature of the method that the delivery of speaking gas takes place in a rectangular profile and/or a decreasing flow profile, with the delivery of speaking gas taking place independently of an exhalation profile of the patient.

The invention further provides a patient interface for at least partially introducing into the trachea of a patient, comprising at least one respiratory tube and at least one speaking tube and at least one sealing element, a gas being able to be conducted through the respiratory tube and the speaking tube independently of each other, the speaking tube being connected via a speaking attachment to a speaking gas line and being at least temporarily supplied with speaking gas, and the respiratory tube being connected via a respiratory attachment with a Y piece to at least one respiratory gas line and being at least temporarily supplied with respiratory gas, wherein the speaking tube has a speaking opening which is arranged between the speaking attachment and the sealing element.

In some embodiments, it is a characteristic feature of the patient interface that the patient interface is a tracheal cannula.

In some embodiments, it is a characteristic feature of the patient interface that the respiratory tube and the speaking tube run parallel to each other at least in sections.

In some embodiments, it is a characteristic feature of the patient interface that the respiratory tube and the speaking tube run parallel at least in sections in a common tube, wherein the respiratory tube and the speaking tube are separated from each other by a tube wall.

In some embodiments, it is a characteristic feature of the patient interface that the speaking tube is closed downstream of the speaking attachment and speaking opening by a tube closure such that speaking gas introduced into the speaking tube via the speaking attachment escapes substantially only via the speaking opening.

In some embodiments, it is a characteristic feature of the patient interface that the sealing element is designed as a balloon and/or cuff, and a balloon line is guided in the tube next to the respiratory tube and the speaking tube.

In some embodiments, it is a characteristic feature of the patient interface that the respiratory tube and the speaking tube are guided into the tube via a Y piece.

In some embodiments, it is a characteristic feature of the patient interface that both the speaking attachment and the respiratory attachment are arranged directly on and/or in the Y piece.

In some embodiments, it is a characteristic feature of the patient interface that a holding plate is arranged on the tube, the holding plate being designed and configured in such a manner that the patient interface can be substantially fixed to the patient's neck.

In some embodiments, it is a characteristic feature of the patient interface that the tube can be at least partially introduced into the trachea of a patient, wherein respiratory gas is conducted via the respiratory tube to and/or from the patient's lungs, the sealing element substantially seals the trachea downstream of the patient's glottis in the direction of the lungs such that no respiratory gas is conducted into the trachea between the sealing element and the glottis, and wherein the speaking opening is arranged between glottis and sealing element such that no speaking gas goes past the sealing element into the patient's lungs.

It will be noted that the features individually presented in the claims can be combined with one another in any desired, technically meaningful way and disclose further refinements of the invention. The description additionally characterizes and specifies the invention in particular in conjunction with the figures.

It will also be noted that an “and/or” conjunction used herein between two features, and linking them to each other, is always to be interpreted as meaning that in a first refinement of the subject matter according to the invention only the first feature may be present, in a second refinement only the second feature may be present, and in a third refinement both the first and the second feature may be present.

A ventilation apparatus should be understood as meaning any apparatus which supports the natural breathing of a user or patient, which takes over the ventilation of the user or living being (e.g. patient and/or neonate and/or premature baby) and/or which serves for respiration therapy and/or influences the respiration of the user or patient in some other way. This includes by way of example, but not exclusively, CPAP and bilevel apparatuses, anesthesia apparatuses, respiratory therapy apparatuses, ventilation apparatuses (for use in hospitals, in non-hospital environments or in emergencies), high-flow therapy apparatuses and coughing machines. Ventilation apparatuses can also be understood as meaning diagnostic apparatuses for ventilation. Diagnostic apparatuses can generally be used to detect medical and/or respiratory parameters of a living being. These also include apparatuses that are able to detect and optionally process medical parameters of patients in combination with respiration or only in relation to respiration.

Unless specifically described otherwise, a patient interface can be understood as meaning any peripheral conceived for interaction of the measuring device with a living being, in particular for therapeutic or diagnostic purposes. In particular, a patient interface can be understood as meaning a mask of a ventilation apparatus or a mask connected to the ventilation apparatus. This mask can be a full-face mask, i.e. enclosing the nose and mouth, or a nose mask, i.e. a mask enclosing only the nose. Tracheal cannulas and so-called nasal cannulas can also be used as a mask or patient interface. A high flow mask can also be understood as meaning a patient interface. In some cases, the patient interface can also be a simple mouthpiece, for example a tube, through which the living being at least exhales and/or inhales. For the invention described here, the patient interface should be understood as meaning in particular tracheal cannulas and, which, unless mentioned explicitly otherwise, are used as synonyms.

It should be pointed out that, in some embodiments of the invention, the respiratory gas and the speaking gas originate from the same source. The respiratory gas and speaking gas then differ in part only when the gas is conducted to the speaking tube and/or to the respiratory tube of the patient interface. In some embodiments, a gas source can be designed to deliver both respiratory gas and speaking gas. For example, two ventilation apparatuses which are both suitable for delivering respiratory gas can be arranged in the inventive system. The gas which is delivered by the ventilation apparatus and which is conducted to the speaking tube of the patient interface is referred to here as speaking gas, but may equally also be suitable as respiratory gas. All in all, it is thus primarily decided by the target of the gas as to whether it is referred to as respiratory gas or speaking gas and not necessarily on the basis of the source. The expression gas, unless indicated otherwise, is used synonymously for respiratory gas and speaking gas.

The speaking gas can be considered to be any gas or gas mixtures which do not have a harmful effect on the human body. In particular, gases and/or gas mixtures are usable as speaking gas and same are generally also usable as respiratory gas. In particular, these include (synthetic) air, for example ambient air and/or compressed air from a central compressed gas line or from a compressed air cylinder, and mixtures of oxygen and nitrogen and/or further gases. In principle, even pure nitrogen which is generally not suitable as respiratory gas is usable as speaking gas.

The inventive system can be realized in various embodiments. In some embodiments, the ventilation apparatus which delivers the respiratory gas for ventilation of the patient also provides the gas which is conducted through the speaking tube of the tracheal cannula and enables the patient to speak. In other embodiments, the system comprises at least one further apparatus, for example a further ventilation apparatus and/or an apparatus via which a gas, which is suitable as speaking gas, can be delivered, in an at least partially controlled manner.

In some exemplary embodiments, the system comprises a ventilation apparatus which operates both the respiratory tube and the speaking tube of the tracheal cannula with respiratory/speaking gas. For this purpose, the ventilation apparatus comprises, for example, a respiratory gas source. The ventilation apparatus is connected via a gas-conducting connection to a Y piece which, in turn, is connected to the respiratory tube of the tracheal cannula and to an exhaled air device of the ventilation apparatus. The ventilation apparatus is configured in such a manner that it identifies the respiratory phases, at least inspiration and expiration, and correspondingly controls the supply of gas. During inspiration, the control is performed in such a manner that respiratory gas is delivered through the connection to the Y piece and into the respiratory tube of the tracheal cannula. During the expiration, for example, the line through which the respiratory gas is delivered to the patient is blocked, and therefore the exhaled air from the patient is conducted through a gas-conducting connection through the Y piece to the exhaled air device. For example, an additional switching valve is arranged in the system, optionally in and/or on the ventilation apparatus. As an alternative thereto, the switching valve can also be arranged in the gas-conducting connection between ventilation apparatus and Y piece, through which connection the respiratory gas is delivered to the patient during the inspiration. This switching valve is connected to the respiratory gas source in such a manner that a delivery of gas to the respiratory tube and/or speaking tube of the tracheal cannula can be switched. During the inspiration of the patient, the switching valve is switched, for example, in such a manner that respiratory gas is delivered to the Y piece and to the respiratory tube. For the expiration, the switching valve is then switched, for example, in such a manner that respiratory gas or speaking gas is delivered instead to the speaking tube of the tracheal cannula. The valve is designed, for example, as a pneumatic switching valve or as an electric switching valve.

In some embodiments, the flow profile of the speaking gas, in particular during the speaking mode, is independent of the expiration profile of the patient. The actual exhalation of the patient is therefore not simulated by the delivery of speaking gas. Instead, provision is made for it to be possible to select at least between a rectangular profile and a decreasing flow profile for the speaking gas flow. The rectangular profile here provides a substantially constant speaking gas flow. If the patient, for example, is enabled to speak only during the expiration, by conduction of speaking gas into the speaking tube, the speaking gas flow is greatly increased at the beginning of the expiration and kept at a constant level until the end of the expiration. At the end of the expiration of the patient, the speaking flow is then reduced again.

The decreasing flow profile for the speaking gas makes provision for a flow of speaking gas to be predetermined at the beginning of the expiration of the patient, said flow decreasing constantly during the expiration. Towards or at the end of the expiration, the speaking gas flow is also reduced here again to 0 and/or to a predeterminable basic level.

In some embodiments, provision can be made for the flow profile of the delivery of speaking gas to be able to be freely configured. For some embodiments, provision can also be made for the speaking gas profile to correspond to a recorded expiration profile of the patient.

For example, the valve is also switchable in such a manner that an at least low pressure and/or flow in the speaking tube can be maintained, even if the patient is in the inspiration phase.

In an alternative or additional embodiment of the system, the system comprises a ventilation apparatus and a second apparatus, called speaking apparatus here, for delivering respiratory gas/speaking gas. The two apparatuses are connected to each other in such a manner that the ventilation apparatus which controls and/or assists the ventilation of the patient also controls the speaking apparatus which delivers the speaking gas. The speaking apparatus can be designed, for example, in such a manner that a constant flow and/or pressure is provided; in this case, for example, switching on/off of the speaking apparatus or of the gas source of the speaking apparatus is controlled by the ventilation apparatus. The speaking apparatus is connected here, for example, directly to the speaking attachment and to the speaking tube of the tracheal cannula. The ventilation apparatus controls the speaking apparatus, for example, in such a manner that the gas source of the speaking apparatus does not deliver any gas to the speaking tube during the inspiration of the patient. During the expiration of the patient, the speaking apparatus or the gas source of the speaking apparatus is then switched on/connected such that speaking gas is delivered to the speaking tube of the tracheal cannula. It may also be envisaged for the gas source of the speaking gas, for example a fan, to permanently maintain a low pressure (e.g. below 5 mbar) and/or flow in the speaking tube and to increase the pressure and/or flow to enable speaking. In such a system, the use of an additional valve for controlling the gas flow to the speaking tube is not necessary.

In some embodiments, a low minimum flow and/or minimum pressure of speaking gas in the speaking tube is maintained by the speaking apparatus irrespective of the speaking of the patient.

In some embodiments, the speaking apparatus is configured in such a manner that the gas source is controllable such that a switch can be made at least between two gas pressure and/or gas flow levels, for example comparably to a bilevel ventilation apparatus. For example, by means of the ventilation apparatus, the speaking apparatus is controlled in such a manner that, during the inspiration of the patient, a low pressure/flow is provided by the speaking apparatus and, during the expiration of the patient, a switch is made to a higher pressure/flow of the speaking apparatus, as a result of which it becomes possible for the patient to speak.

The low pressure of the speaking apparatus during the inspiration of the patient is selected in such a manner that the patient can close the glottis easily and without leakage and can keep it closed. For example, the pressure for this purpose is under about 5 mbar, preferably in a range from about 1 mbar to about 3 mbar. The second apparatus, i.e. the speaking apparatus, can also be designed in such a manner that it can be coupled to the ventilation apparatus, for example via a serial interface.

In some embodiments, in particular if the speaking apparatus provides a constant flow/pressure, there is the possibility of arranging an additional valve between the speaking apparatus and speaking attachment of the tracheal cannula. This valve is configured, for example, in such a manner that, depending on the respiratory phase of the patient, a gas flow is conducted to the speaking attachment/to the speaking tube of the tracheal cannula. If no speaking gas is needed in the speaking tube, the valve is switched in such a manner that the speaking gas flow can escape, for example, into the ambient air. For example, a pneumatic valve which is switched via an electric/proportional switching valve is envisaged here. The compressed air for controlling the pneumatic valve is provided, for example, via the gas source of the speaking apparatus.

In some embodiments of the system, the speaking apparatus is independent of the ventilation apparatus. The speaking apparatus is configured here in such a manner that it can identify the respiratory phases of the patient independently of the ventilation apparatus and accordingly delivers speaking gas to the speaking tube of the tracheal cannula. For example, the speaking apparatus has for this purpose a pressure and/or flow sensor, with the signal or the measured values being tapped off at the Y piece. A flow and/or pressure sensor can also be arranged on the Y piece, the signals of which are taken off at least from the speaking apparatus. The speaking apparatus controls the delivery of speaking gas to the speaking tube in accordance with the respiratory phase.

In some embodiments of the inventive system, it is envisaged that the speaking apparatus has a standby mode. In the standby mode, a low pressure and/or flow of speaking gas is permanently maintained by the speaking apparatus. The pressure is regulated here, for example, in such a manner that the patient can keep the glottis slightly closed, for example below about 5 mbar, preferably at maximum about 3 mbar. If the patient begins to speak or would like to speak, the apparatus can change from the standby mode into an assisting speaking mode and, by increasing the pressure/the flow, can permit or assist the patient to speak.

In some embodiments of the system, the control of the speaking gas delivery is based, in addition or alternatively to identification of the respiratory phases of the patient, on an identification of an intention of the patient to speak and/or of an attempt of the patient to speak. For example, a small flow/pressure is permanently provided to the speaking tube via the speaking apparatus, e.g., in a range of from about 1 mbar to about 5 mbar, so that the patient can keep the glottis closed. This corresponds, for example, to a standby mode or standby state. For example, sensors corresponding to the identification of speaking are arranged in the speaking tube and/or on the speaking attachment. For example, it can be identified via an acceleration sensor in the speaking tube whether the patient is speaking or would like to speak. Alternatively, a microphone can also be used here to identify whether the patient is speaking. If the intention to speak/speaking is identified, the flow/pressure of the speaking gas is correspondingly increased by the speaking apparatus. An identification of speaking is also conceived via a flow and/or pressure sensor which measures the pressure/flow through the speaking tube. If the patient begins to speak, the pressure decreases (or the flow increases), i.e. the speaking apparatus can correspondingly also provide the necessary flow. If the patient stops speaking, the pressure increases or the flow decreases, and, accordingly, the speaking apparatus can control the flow/pressure again to a standby value.

In addition, it is also conceivable for the speaking apparatus to be integrated in the ventilation apparatus. The ventilation apparatus here can be divided, for example, into a ventilation module and a speaking module. The speaking module comprises, for example, at least one gas source from which speaking gas is delivered to the speaking tube of the tracheal cannula when required. The ventilation module is configured, for example, in such a manner that it completely takes over the control from the speaking module. Alternatively thereto, the speaking module is configured as an independent module such that, for example, only the supply of energy and/or information regarding the respiratory phases of the patient are supplied from the ventilation module. In some embodiments, the speaking module can be at least switched on and off via the ventilation module.

It is furthermore envisaged that the level of the speaking gas flow is adjustable, for example in order to be able to regulate the loudness of the speaking. A higher speaking gas flow leads, for example, to a louder voice, while a smaller speaking gas flow results in a quieter voice. It should be noted here that a certain threshold value for the speaking gas flow has to be exceeded so that an audible voice/speaking of the patient arises. This threshold value is dependent in particular on the patient's physiology.

In some embodiments, it is also envisaged that the respiratory gas and/or the speaking gas is moistened and/or heated, optionally independently of each other. For this purpose, at least one respiratory gas moistener and/or heater is arranged in the system. The respiratory gas moistener and/or the heater can be integrated in the ventilation apparatus and/or the speaking apparatus. Coupling to the ventilation apparatus and/or to the speaking apparatus, for example via the respiratory gas line and/or speaking gas line, is also conceivable.

The invention also provides to a method for controlling the delivery of speaking gas to a user/patient. In one method step, in order to assist speaking or in a speaking mode, the speaking gas flow and/or speaking gas pressure is regulated to a first value which enables a user to speak. In a further method step, the speaking gas flow and/or speaking gas pressure is regulated in a standby mode to a second, lower value which permits the user to at least partially and/or for the most part close the glottis. In some embodiments, the switching over from standby mode into the speaking mode requires the identification of an intention to speak by the user. The identification of the intention to speak can take place, for example, via detecting a drop in the speaking gas pressure and/or a rise in the speaking gas flow during the standby mode.

The flow profile of the delivery of speaking gas is, for example, independent of the flow profile of the expiration of the patient. For delivering the speaking gas, a selection can be made, for example, from various predefined flow profiles. Provision is made, inter alia, for the delivery of speaking gas to take place in a rectangular profile. At the beginning of the expiration of the patient, the speaking gas flow is raised to a flow level which remains substantially constant during the speaking and/or the expiration of the patient. Toward and/or at the end of the expiration, the flow of speaking gas is then reduced again. Alternatively or additionally, a decreasing flow profile can be provided for the delivery of speaking gas, during which, at the beginning of the expiration of the patient, the speaking gas flow is raised to a flow level and gradually decreases. Toward and/or at the end of the expiration of the patient, the speaking gas flow is then completely lowered again. The speaking gas flow profiles can optionally be at least partially adaptable, for example in the level of the flow and/or degree of the decrease. In some embodiments, provision can also be made for the flow profile of the delivery of speaking gas to be able to be freely configured.

### BRIEF DESCRIPTION OF THE DRAWINGS

The invention is described in more detail by way of example with reference to FIGS. 1 to 13. In FIGS. 1 to 8, exemplary embodiments of the system and the functioning thereof are described in more detail and FIGS. 9 to 13 illustrate exemplary embodiments of the patient interface. Specifically,

FIG. 1 schematically illustrates an exemplary system according to the present invention;

FIG. 2 schematically illustrates a second exemplary system according to the present invention;

FIG. 3 schematically illustrates a third exemplary system according to the present invention;

FIG. 4 schematically illustrates a fourth exemplary system according to the present invention;

FIG. 5 schematically illustrates a fifth exemplary system according to the present invention;

FIG. 6 schematically illustrates a sixth exemplary system according to the present invention;

FIG. 7 illustrates the time curve of the flow of the respiratory gas provided by the systems of FIGS. 1, 2 and 4 to 6;

FIG. 8 shows exemplary flow or pressure curves of the speaking gas or the respiratory gas;

FIG. 9 shows an exemplary embodiment of a patient interface according to the invention;

FIGS. 10 to 12 show an exemplary embodiment of a patient interface according to the invention in the form of a tracheal cannula; and

FIG. 13 shows another exemplary embodiment of a patient interface according to the invention in the form of a tracheal cannula.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

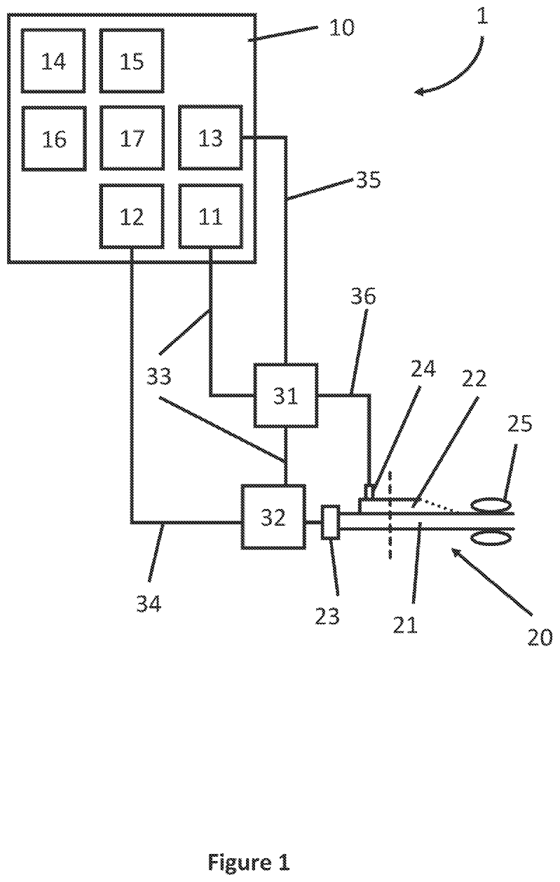

An exemplary embodiment of the system 1 can be seen in FIG. 1. In this embodiment, the system 1 comprises a ventilation apparatus 10 which is connected in a gas-conducting manner to the patient interface 20. The patient interface 20 is designed by way of example as a tracheal cannula. The patient interface 20 comprises at least one respiratory tube 21 which is at least partially introduced into the patient's trachea. The trachea is sealed around the respiratory tube 21 via at least one sealing element 25. The sealing element 25 ensures that respiratory gas is conducted to and from the patient's lungs only or primarily by the respiratory tube 21. The sealing element 25 is designed by way of example as a balloon or balloon sleeve or as what is referred to as a cuff around the respiratory tube 21. In addition to the respiratory tube 21, the patient interface 20 also comprises a speaking tube 22. The speaking tube 22 is integrated by way of example in the tracheal cannula and runs at least partially parallel to the respiratory tube 21. The speaking tube 22 is designed, for example, in such a manner that it has an opening which, for example, is positioned just behind the patient's glottis but in front of the sealing element 25. The speaking gas is conducted through the speaking tube 22 and can escape into the oral cavity through the glottis, in particular whenever the patient is speaking or would like to speak. The speaking gas simulates an expiration of the patient through the glottis when speaking. The speaking tube 22 is connected via the speaking attachment 24 to a gas-conducting connection, illustrated as a speaking gas line 36 in FIG. 1, via which the speaking gas is conducted to the speaking tube 22. The respiratory tube 21 is connected in a gas-conducting manner to a Y piece 32 via a respiratory attachment 23.

The system 1 is configured and designed in such a manner that the system 1 can switch at least temporarily into a speaking mode 63 in which speaking gas is made available to the patient via the speaking tube 22 and enables the patient to speak.

Via the Y piece 32, respiratory gas is conducted to the respiratory tube 21 during the inspiration of the patient and is conducted away from the respiratory tube 21 during the expiration of the patient. During the expiration, the patient can therefore exhale through the speaking tube 21 and the Y piece 32.

The ventilation apparatus 10 comprises at least one respiratory gas source 11, an exhaled air device 12, a speaking controller 13, a control unit 14, a sensor unit 15, an evaluation unit 16, and a user interface 17. The ventilation apparatus 10 is configured to at least partially also comprise the features of a speaking apparatus 40 or to act as a combined ventilation apparatus and speaking apparatus. The ventilation apparatus 10 is configured by way of example to deliver both speaking gas and respiratory gas.

The controllable respiratory gas source 11 is designed to at least temporarily deliver respiratory gas to a patient. The respiratory gas source 11 can be, for example, a fan unit and/or valve unit which generates the respiratory gas from the ambient air and/or gas cylinders. In some embodiments, the delivered respiratory gas is conducted through at least one filter, for example in order to at least partially filter dust, germs, pathogens, etc. out of the respiratory gas. In the embodiment of the system 1 that is illustrated in FIG. 1, the gas delivered by the respiratory gas source 11 is used both as respiratory gas and as speaking gas. In some embodiments, the ventilation apparatus 10 furthermore also comprises devices for conditioning the respiratory gas, for example for moistening and/or for heating same. This can alternatively or additionally also be achieved via a corresponding module which is coupled to the ventilation apparatus 10 or is integrated in the system 1 as an additional respiratory gas moistener.

The control unit 14 serves, for example, for controlling the ventilation apparatus 10, in particular the respiratory gas source 11 and the valve controller 13. The control unit 14 is designed, inter alia, to control the pressure and/or flow generated by the respiratory gas source 11. The control unit 14 can also be designed to control other constituent parts/components of the ventilation apparatus 10. In some embodiments, the control unit 14 can also be further divided and can consist of a plurality of control units which each control an individual unit and/or constituent part of the ventilation apparatus 10. In particular, the control unit 14 is configured to at least partially automatically control the ventilation apparatus 10. In particular, the control unit 14 is programmable in such a manner that various maneuvers, during which at least one ventilation setting is changed, can be carried out.

The sensor unit 15 is configured to detect measured values, in particular parameters, which are associated with a respiratory flow, a respiratory volume, a respiratory frequency, an inspiration and/or expiration duration, a respiratory contour, a leakage and/or a therapy or respiratory gas pressure. For this purpose, the sensor unit 15 comprises at least one correspondingly configured sensor or is connected to a correspondingly configured sensor. In particular, the sensor unit 15 is configured to detect the respiratory gas pressure and/or the respiratory gas flow. The sensor unit 15 can optionally also be configured to detect further parameters/measured values, such as, for example, temperature and/or oxygen concentration and/or CO₂ concentrations of the respiratory gas and/or of the blood. A coupling to further and/or external sensors and/or sensor units, for example a pulse oximeter, an EIT device, etc., is also possible in some embodiments.

The parameters and/or measured values detected by the sensor unit 15 are conducted, for example, to the evaluation unit 16. The evaluation unit 16 is designed, for example, as a combined conditioning, calculation, identification and evaluation unit. The evaluation unit 16 is configured, for example, to condition and to evaluate the measured values and/or parameters detected by, for example, the sensor unit 15.

For example, the evaluation unit 16 can perform smoothing, artefact cleanup and/or down-sampling of the measured values. Furthermore, the evaluation unit 16 is designed to calculate, from the conditioned measured values, signals and/or characteristic quantities, such as for example a mean value, a median, a percentile, a derivative, a frequency distribution, a duration and/or a proportion of values above or below threshold values. The evaluation unit 16 is furthermore configured to identify events/states such as for example alarms, respiratory dropouts, artefacts, bursts of coughing, oxygen (de)saturations, asynchronous states between apparatus and user, and/or spontaneous breaths. In particular, the evaluation unit 16 is configured to identify the respiratory phases of the patient, but at least the inspiration and/or expiration. In some embodiments, the evaluation unit 16 is also configured to detect technical problems with the ventilation apparatus 10. Technical problems can be for example a low battery level, errors in the electronics, a defective battery, a defective component, a power failure, an incorrectly functioning accessory, an implausible measured value or a departure from a permitted temperature range. The evaluation unit 16 can display and/or transmit an alarm on the ventilation apparatus 10 and/or by way of an interface in the event of a detected technical problem.

In some embodiments, the ventilation apparatus 10 also comprises a storage unit, not illustrated here. This storage unit is configured, for example, to store and/or to temporarily store data and/or values and/or information. Said data/values/information can be, for example, the values/data/information detected by the sensor unit 15 and/or conditioned by the evaluation unit 16. For example, values/data/information can also be stored in the storage unit, for example by being input via a user interface 17.

User inputs, for example settings for the ventilation and/or selection of a ventilation program, can be input, for example, via the user interface 17. The user interface 17 is in the form of a touchscreen, for example. In some embodiments, as an alternative or addition thereto, buttons and/or switches and/or rotary regulators, optionally together with a display, can also be arranged as the user interface 17.

The exhaled air device 12 is configured, for example, in such a manner that the exhaled respiratory gas from the patient is conducted away here into the surroundings. For this purpose, the exhaled air device 12 is connected to the Y piece 32, for example, via the respiratory gas line 34. For example, the exhaled air device 12 can be at least partially controlled via the valve which also controls the pressure/flow of the respiratory gas source 11. In some embodiments, the exhaled respiratory gas in the exhaled air device 12 is filtered and/or cleaned in some other way prior to escaping into the ambient air.

The ventilation apparatus 10 is configured, for example, depending on the respiratory phase of the patient, to raise the pressure and/or the flow of the respiratory gas to an inspiratory level in order to assist and/or to predetermine the inspiration of the patient. In order to assist and/or to predetermine the expiration of the patient, the ventilation apparatus 10 is configured to lower the respiratory gas flow and/or respiratory gas pressure to an expiratory level. In some embodiments, during the expiration, the respiratory gas line 33 is blocked in such a manner that essentially no respiratory gas flows from the Y piece 32 through the respiratory gas line 33. In some embodiments, the pneumatics inside the ventilation apparatus 10 are configured and designed in such a manner that the respiratory gas source 11 during the expiration can maintain a respiratory gas pressure, for example a positive end-expiratory pressure (PEEP) and/or an expiratory positive airway pressure (EPAP), in the respiratory gas line 34.

In addition, during the expiration, the intention is to enable the patient to speak. For this purpose, the system 1 performs control in such a manner that respiratory gas or speaking gas is delivered to the speaking tube 22 of the patient interface 20. By way of example, the same gas which is delivered by the respiratory gas source 11 as respiratory gas is used as speaking gas. In order to make respiratory gas or speaking gas available to the speaking tube 22 during the expiration of the patient, a control valve 31 is arranged in the respiratory gas line 33 between the respiratory gas source 11 and the Y piece 32. The control valve 31 is configured to conduct, during the inspiration of the patient, the gas flow to the Y piece 32 and therefore at least partially to the respiratory tube 21. During the expiration of the patient, the control valve 31 can be switched in such a manner that a gas flow to the speaking tube 22 of the patient interface 20 is possible. The speaking controller 13 is configured to control the control valve 31.

The control valve 31 can be designed, for example, as a pneumatic and/or as an electric valve and is connected to the speaking controller 13 via the control line 35. The speaking controller 13 is controlled, for example, via the control unit 14. In some embodiments, the speaking controller 13 is also designed to automatically identify whether a control valve 31 is connected to the ventilation apparatus 10.

If an expiration of the patient is identified and/or predetermined by the ventilation apparatus 10, the switching valve 31 is switched by the control unit 14 or the speaking controller 13 in such a manner that the gas delivered from the respiratory gas source 11 is at least partially conducted to the speaking tube 22 and enables the patient to speak. If the inspiration or a respiratory endeavor of the patient is identified and/or predetermined, the control valve 31 is again switched in such a manner that the respiratory gas is conducted to the respiratory tube 21.

The control valve 31 can also be controlled, for example, in such a manner that speaking gas is only conducted via the speaking gas line 36 to the speaking tube 22 whenever an intention of the patient to speak is identified. In addition to various sensory possibilities, for example microphones and/or acceleration sensors in the speaking tube 22, a switch in the vicinity of the patient is also conceivable for this purpose. If such a switch, which is connected, for example, directly to the control valve 31 or to the ventilation apparatus 10, is actuated, the control valve 31 is opened with respect to the speaking gas line 36 and speaking gas is conducted to the speaking tube 22 such that the patient can speak.

In some embodiments, the ventilation apparatus 10 is configured in such a manner that the control valve 31 is controlled such that it permits a gas flow to the speaking tube during the expiration of the patient, but the provided gas flow and/or gas pressure is adjusted downward to such an extent that the patient can keep the glottis closed, for example is adjusted downward to a pressure below 5 mbar, preferably between 1 mbar and 3 mbar. If the patient starts to speak, the glottis is at least partially opened, whereupon a drop in pressure and/or an increase in flow in the speaking tube can be or will be detected. The provided gas flow and/or gas pressure through the speaking tube can thereupon be adjusted upward to such an extent that the patient is enabled to speak. For example, an adjustment is made here to a gas flow which is in a range from 20 l/min to 60 l/min, preferably in a range from 30 l/min to 50 l/min.

In some embodiments, the speaking controller 13 is designed as a software and/or firmware module via which certain components of the ventilation apparatus 10 can be activated. For example, it can be possible that the speaking controller 13 is used to activate an electric signal transmitter which is connected via a control line 35 to the control valve 31, which is designed for example as an electric switching valve. Alternatively, a pneumatic control unit of the ventilation apparatus 10 can also be activated, said ventilation apparatus activating a control valve 31 designed as a pneumatic switching valve.

In some embodiments, the speaking controller 13 can also be designed as a module which can be coupled to the ventilation apparatus 10 and which receives the control signals for controlling the control valve 31 via the ventilation apparatus 10, for example from the control unit 14, and thereupon appropriately controls the switching valve 31.

In some embodiments, the ventilation apparatus 10 has a switchable speaking function. If the speaking function is switched on, the controller of the switching valve 31 is activated via the speaking controller 13 and the patient is enabled to speak during the expiration. The speaking function can be activated and deactivated, for example, via the user interface 17 of the ventilation apparatus 10. In some embodiments, the speaking function is activated automatically when the ventilation apparatus 10 identifies a connected control valve 31. The switching on of the speaking function enables the system to switch into the speaking mode in which the patient is enabled to speak by delivery of speaking gas to the speaking tube 22.

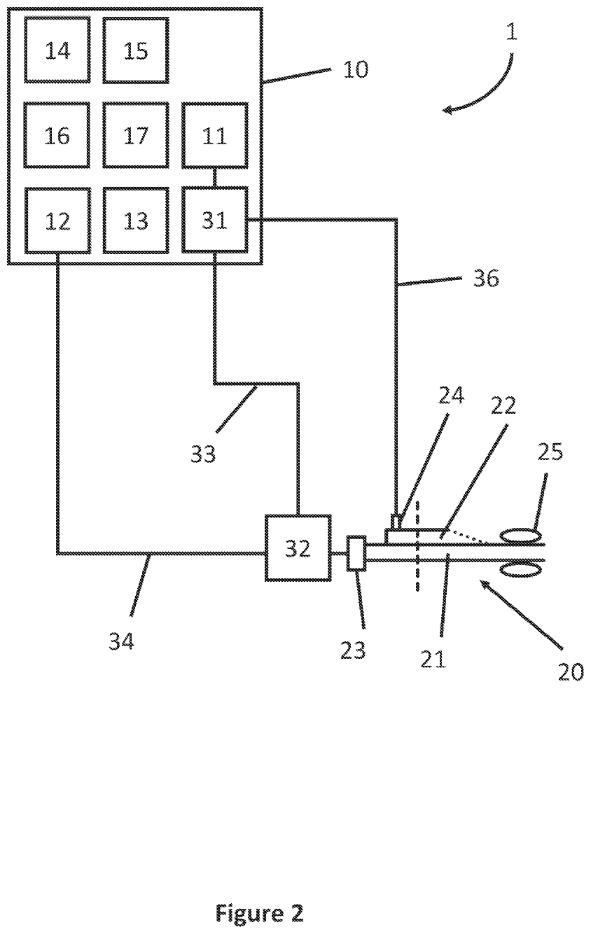

An exemplary embodiment of the system 1, in which the control valve 31 is integrated in the ventilation apparatus 10, is illustrated schematically in FIG. 2. The ventilation apparatus 10 is substantially configured in the same manner as the embodiment described in FIG. 1 and differs in particular in that the control valve 31 is arranged inside the ventilation apparatus 10. The switching valve 31 is controlled in accordance with the same specifications as explained for FIG. 1. During the expiration of the patient, the switching valve 31 is switched, for example by the speaking controller 13, in such a manner that a respiratory or speaking gas flow is conducted via the speaking gas line 36 to the speaking tube 22 of the patient interface 20. This enables the patient to speak during the expiration phase. By contrast, for the inspiration, the control valve 31 is switched in such a manner that the respiratory gas is conducted via the respiratory gas line 33 to the Y piece 32 and therefore to the respiratory tube 21 of the patient interface 20. In some embodiments, the ventilation apparatus 10 is configured in such a manner that the gas flow and/or gas pressure for the inspiration and the speaking is respectively adapted during the expiration. In some embodiments, the respiratory gas pressure and/or respiratory gas flow is identical to the speaking gas pressure and/or speaking gas flow.

The system 1 is configured and designed in such a manner that the system 1 can at least temporarily switch into a speaking mode 63 in which speaking gas is made available to the patient via the speaking tube 22 and enables the patient to speak.

The ventilation apparatus 10 takes on, for example in combined form, the function of a ventilation apparatus and of a speaking apparatus. For this purpose, it is at least designed to deliver both respiratory gas and speaking gas. In some embodiments, the ventilation apparatus 10 is designed in such a manner that respiratory gas and speaking gas are delivered simultaneously.

In some embodiments (not illustrated), the switching valve 31 can also be integrated in the Y piece 32.

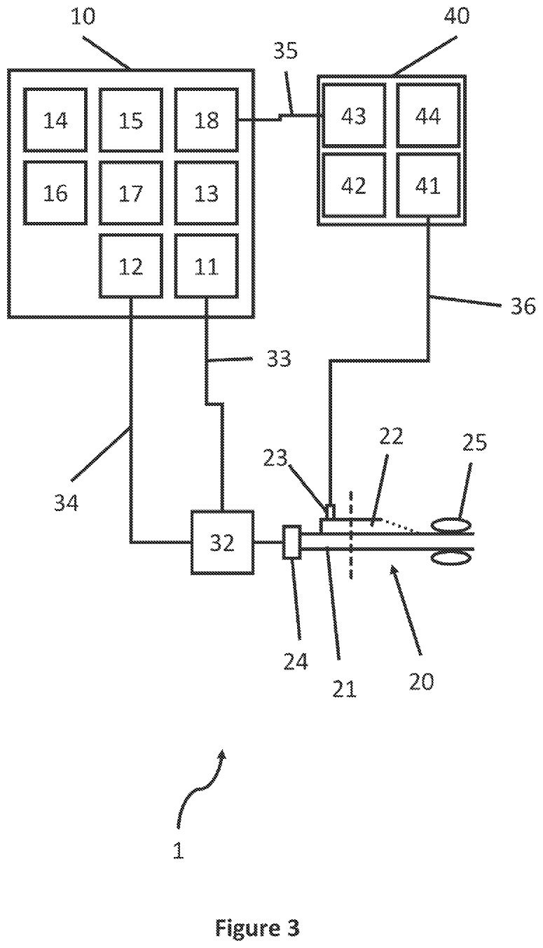

FIG. 3 schematically shows an exemplary embodiment of the system 1, wherein the system 1 in addition to the ventilation apparatus 10 comprises a speaking apparatus 40 which is configured to deliver speaking gas to the speaking tube 22 of the patient interface 20. The ventilation apparatus 10 substantially corresponds to the embodiment which is described in

FIG. 1, and possible differences will be correspondingly discussed. The system 1 is configured and designed in such a manner that the system 1 can switch at least temporarily into a speaking mode 63 in which speaking gas is made available to the patient via the speaking tube 22 and enables the patient to speak.

The speaking apparatus 40 comprises at least one controllable gas source 41, a control unit 42, and an interface 43. The embodiment illustrated by way of example also additionally comprises a user interface 44, for example in the form of a touchscreen and/or display with operator control elements. The gas source 41 is configured to deliver a speaking gas. For example, the gas source 41 can be a fan device and/or valve device and can suck up ambient air as speaking gas and/or can deliver a gas or a gas mixture from a compressed gas source (e.g., central gas attachment and/or gas cylinders). The control unit 42 is configured to control at least the gas source 41. The interface 43 is configured to construct a connection to the ventilation apparatus 10, wherein the connection can be, for example, a cable connection or else a wireless connection, for example a radio connection. In the embodiment illustrated by way of example in FIG. 3, the interface 43 of the speaking apparatus 40 is connected via a control line 35 to an interface 18 of the ventilation apparatus 10. The user interface 44 is configured to be able to input inputs from a user at the speaking apparatus 40. The user interface 44 is configured, for example, such that at least the speaking apparatus 40 can be activated/deactivated and/or can be switched on and off via the user interface 44.

By way of example, the gas source 41 is designed as a fan, i.e., the delivery of speaking gas can be controlled exclusively by control of the fan without a valve being required between fan and speaking tube 22. During the phases in which no speaking gas is conducted in or to the speaking tube 22, the fan is deactivated. Provision can also be made for a constant flow and/or pressure to be maintained in the speaking tube 22, even if the system 1 is not in a speaking mode 63. Between speaking mode 63, i.e., the period of time in which speaking gas is conducted into the speaking tube 22, in order to enable the patient to speak, and the period of time outside the speaking mode 63, the fan is not activated or deactivated, but rather the delivery rate is increased or reduced.

The speaking apparatus 40 is connected via the speaking gas line 36 to the speaking tube 22 of the patient interface 20. Speaking gas is delivered via the gas source 41 and conducted through the speaking gas line 36 to the patient interface 20 in order to enable the patient to speak at least temporarily. The delivery of speaking gas through the speaking apparatus 40 is controlled or determined by the ventilation apparatus 10. That is to say that the ventilation apparatus 10 predetermines when and/or what quantity of speaking gas is delivered through the speaking apparatus 40 and/or is conducted to the speaking tube 22. For this purpose, the ventilation apparatus 10 is connected to the speaking apparatus 40 via the control line 35 which, alternatively or additionally, may also be a wireless connection. The control unit 42 of the speaking apparatus 40 is configured to appropriately convert the control signals of the ventilation apparatus 10 and to control the gas source 41 in accordance with the specifications of the ventilation apparatus 10.

The specifications of the ventilation apparatus 10 can be adapted in accordance with the design of the speaking apparatus 40. In the case of a speaking apparatus 40 which is configured merely to provide a constant, optionally predeterminable gas pressure and/or gas flow, the ventilation apparatus 10 can predetermine, for example, that the gas source 41 is activated during the expiration of the patient and delivers speaking gas. If the gas source 41 is designed, for example, as a fan, the delivery of speaking gas can be controlled by switching on/off the fan. In the case of a gas source 41 in the form of a valve device, opening/closing of the valve device is, for example, one possible control variant. For example, in some embodiments, the speaking apparatus 40 is a CPAP ventilation apparatus.

In some embodiments, the speaking apparatus 40 is not controlled directly by the ventilation apparatus 10, but rather information regarding the respiratory phases, in particular inspiration and expiration, is passed on from the ventilation apparatus 10 to the speaking apparatus 40, whereupon the speaking apparatus 40 automatically activates a supply of speaking gas during the expiration of the patient.

In some embodiments, the speaking apparatus 40 is configured and designed in such a manner that a variable speaking gas flow can be generated. For example, the speaking apparatus 40 is designed essentially to switch over between two gas pressure and/or gas flow levels, similarly to a bilevel ventilation apparatus. For example, in some embodiments, the speaking apparatus 40 is a bilevel ventilation apparatus. The ventilation apparatus 10 transmits information regarding the respiratory phases of the patient via the control line 35. During the expiration of the patient, the control apparatus 40 activates the gas source 41 in such a manner that a certain flow of speaking gas through the speaking tube 22 is achieved such that the patient is enabled to speak. For the inspiration of the patient, the speaking apparatus 40 adjusts the speaking gas flow and/or the speaking gas pressure downward to such an extent that the patient can easily close the glottis and/or can easily keep it closed and/or can at least partially or for the most part close the glottis. For example, during the inspiration of the patient, the speaking gas pressure is adjusted to below 5 mbar, preferably to a value of between 1 mbar and 3 mbar.

In particular in the case of an embodiment of the speaking apparatus 40 with a controllable gas source 41 which is configured to switch over between at least two pressure and/or flow levels, a type of standby mode and a speaking mode are possible. For this purpose, for example, a fan can be used as the gas source 41. In the standby mode, a substantially constant low pressure is predetermined in the speaking tube 22 by the speaking apparatus 40 such that the patient can keep the glottis at least partially closed. The speaking apparatus 40 is furthermore configured to ascertain an endeavor/intention of the patient to speak, for example via corresponding sensor units and evaluation units. For example, an endeavor of the patient to speak can be established by the fact that the patient opens the glottis in order to speak and, as a result, an abrupt drop in pressure occurs in the speaking tube 22, the drop in pressure being recorded by the speaking apparatus 40. If an intention to speak is established, the speaking apparatus 40 is configured to change from the standby mode into the speaking mode. For the speaking mode, the speaking apparatus 40 adjusts the pressure and/or flow of the speaking gas upward to such an extent that the patient is enabled to speak. For example, the switching over from the standby mode into the speaking mode is limited to the expiration of the patient. During the inspiration of the patient, the speaking apparatus 40 therefore then cannot switch over from the standby mode to the speaking mode and cannot provide an increased speaking gas flow and/or pressure in order to enable the patient to speak. In embodiments in which the ventilation apparatus 10 takes over the entire control for the speaking apparatus 40, the intention to speak can also be determined, for example, by the ventilation apparatus 10.

The flow profile generated by the gas source 41 during the speaking mode 63 can be, for example, a rectangular profile. For this purpose, in order to enable the patient to speak, the flow of the speaking gas, for example at the beginning of the expiration of the patient, is raised, for example to a previously defined value which then remains substantially constant throughout the period of time of the expiration of the patient. Toward and/or at the end of the expiration of the patient, the flow value of the speaking gas flow is then lowered again. In addition to a constant speaking gas flow during the expiration of the patient or the period of time of the speaking mode 63, a decreasing speaking gas flow can also be provided and/or selectable. The speaking gas flow should be considered as being independent of the flow profile of the expiration of the patient. The flow profile of the expiration of the patient is therefore not imitated or simulated by the flow profile of the delivery of the speaking gas.

In addition to the transmission of control signals and/or information regarding the respiratory phases of the patient from the ventilation apparatus 10 to the speaking apparatus 40, a supply of energy to the speaking apparatus 40 via the control line 35 is also conceivable. In some embodiments, the speaking apparatus 40 is couplable to the ventilation apparatus 10, for example the speaking apparatus 40 can be plugged onto the ventilation apparatus 10, for example as an additional module.

The ventilation apparatus 10 is, for example, configured and designed in such a manner that the speaking function can be activated and deactivated via the user interface 17. When the speaking function is activated, the speaking apparatus 40 is switched on and/or the ventilation apparatus 10 controls the speaking apparatus 40. In some embodiments, the speaking apparatus 40 is always switched on when it is connected to the ventilation apparatus 10. In order to deliver speaking gas, for example in the speaking mode, first of all the speaking function has to be activated via the ventilation apparatus 10. For example, it can thus be selected whether basically speaking assistance is desired or not desired. In some embodiments, the speaking function is automatically activated as soon as the ventilation apparatus 10 identifies that a speaking apparatus 40 is connected. For example, manual deactivation of the speaking apparatus 40 is not prevented.

Furthermore, provision can also be made for the speaking apparatus 40 to independently determine the beginning and the end of the expiration of the patient, for example via sensors and an evaluation unit which is arranged in the speaking apparatus 40 and is designed to correspondingly evaluate the sensor data.

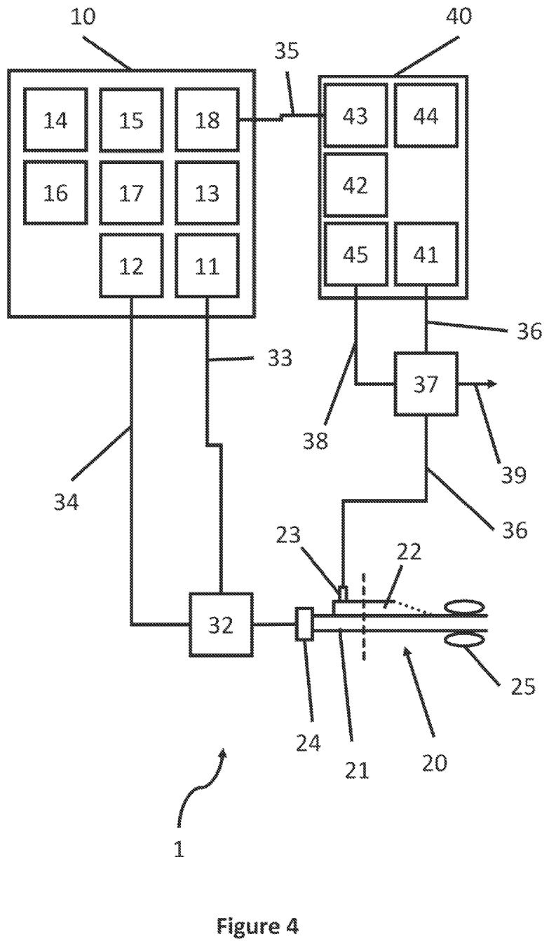

FIG. 4 schematically illustrates a further exemplary embodiment of the system 1. The system 1 is substantially configured in the same manner as the embodiment described in FIG. 3, a control valve 37 being arranged in the speaking gas line 36 between speaking tube 22 and speaking apparatus 40. This control valve 37 can be used to control, for example, the speaking gas flow, for example in the form of pressure and/or flow. For this purpose, the control valve 37 is connected by way of example via a control line 38 to a valve controller 45 in the speaking apparatus 40. The control valve 37 can be designed, for example, as a pneumatic valve, wherein the control pressure which is conducted via the control line 38 to the control valve 37 is generated by the gas source 41 and set via the valve controller 45. The valve controller 45 here comprises a proportional valve, for example. During the expiration of the patient, the control valve 37, for example in the form of a 3/2-way valve, can be switched in such a manner that speaking gas is conducted to the speaking tube 22 of the patient interface 20. During the inspiration of the patient, the control valve 37 is then switched in such a manner that the speaking gas at least partially escapes via the outgoing air 39 into the environment. The system 1 is configured and designed in such a manner that the system 1 can switch at least temporarily into a speaking mode 63 in which speaking gas is made available to the patient via the speaking tube 22 and enables the patient to speak.

The valve controller 45 is controlled, for example, via the speaking controller 13 of the ventilation apparatus 10, i.e., an indirect control of the control valve 37 via the speaking controller 13 is possible. For example, the control commands are generated/output by the speaking controller 13, are transmitted to the speaking apparatus 40 via the interface 18 and are used by the valve controller 45.

The embodiment illustrated in FIG. 4 is, for example, an alternative or additional control form in a system which comprises both a speaking apparatus 40 and a ventilation apparatus 10. The speaking apparatus 40 is configured and designed, for example, in such a manner that a substantially constant speaking gas flow is generated. Instead of deactivating the gas source 41 during the inspiration and of activating same during the expiration, a constant operation of the gas source 41 is possible by means of the control valve 37, with it being controlled via the control valve 37 whether speaking gas is conducted to the speaking tube.

In some embodiments, the control valve 37 is integrated in the speaking apparatus 40. In some embodiments, the control valve 37 is also controlled directly via the ventilation apparatus 10, for example via the speaking controller 13. If the control valve 37 is controlled via the ventilation apparatus 10 or the speaking controller 13, the use of a simple speaking apparatus 40 which provides a constant pressure/flow is possible. The supply of speaking gas to the speaking tube 22 is then regulated exclusively via the ventilation apparatus 10 and the control valve 37.

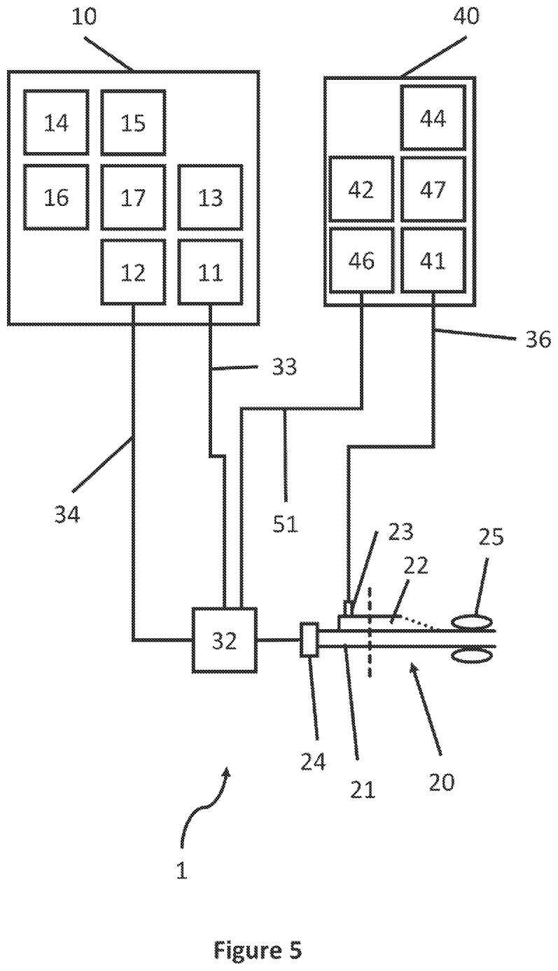

A further exemplary embodiment of the system 1 is illustrated in FIG. 5. The ventilation apparatus 10 and the speaking apparatus 40 are essentially constructed and designed as described in the previously described embodiments. In contrast to the previously described embodiments of the system 1, the speaking apparatus 40 is independent of the ventilation apparatus 10. The ventilation apparatus 10 therefore, for example, does not transmit any data and/or information regarding the respiratory phases or controls the speaking apparatus 40 in some other way. In the embodiment illustrated in FIG. 5, the delivery of speaking gas to the speaking tube 22 of the patient interface 20 is not controlled by the ventilation apparatus 10 either. The speaking apparatus 40 which is independent of the ventilation apparatus 10 is configured, for example, in such a manner that a speaking function can be activated and deactivated. When a speaking function is activated, the speaking apparatus 40 is basically enabled to switch into the speaking mode and to enable the patient to speak. In some embodiments, deactivation of the speaking function also switches off the standby mode.

The speaking apparatus 40 is configured and designed in such a manner that it determines the respiratory phases of the patient, by way of a sensor unit 46 for example. For this purpose, the sensor unit 46 accesses pressure and/or flow data of the respiratory gas via the Y piece 32. For this purpose, in some embodiments, a pressure and/or flow sensor is arranged in the Y piece 32 and is connected by the sensor connection 51 to the sensor unit 46 of the speaking apparatus 40. In some embodiments, the pressure and/or flow sensor is arranged in the speaking apparatus 40 itself such that a pneumatic connection to the Y piece 32 is produced via the sensor connection 51.

The speaking apparatus 40 automatically determines, for example via a dedicated internal evaluation unit 47, which is at least partially comparable to the evaluation unit 15 of the ventilation apparatus 10, the respiratory phases of the patient—irrespective of whether they are predetermined by the ventilation apparatus 10 or are based on an endeavor of the patient to breathe. On the basis of the recorded respiratory phases, the speaking apparatus 40 automatically controls the delivery of speaking gas. For example, during the expiration phase, a speaking gas flow and/or pressure enabling the patient to speak is provided. By contrast, during the inspiration of the patient, for example, the flow and/or pressure of speaking gas is adjusted downward to such an extent that the patient can at least partially or for the most part close the glottis or keep it closed. In some embodiments, during the inspiration of the patient, such a small flow and/or pressure of speaking gas is predetermined by the speaking apparatus 40 that the patient may possibly not be able to completely close the glottis but nevertheless an unpleasant sensation does not arise. For example, the speaking gas pressure is adjusted to below about 5 mbar, preferably below about 3 mbar but above about 1 mbar, during the inspiration of the patient.

A standby mode can also be provided in the embodiment illustrated in FIG. 5. For this purpose, the speaking apparatus 40 during the standby mode predetermines a low pressure and/or flow of speaking gas. If the patient opens the glottis in order to speak, the speaking apparatus 40 is configured to record the drop in pressure and/or increase in flow and, for example after an optionally adjustable threshold value is exceeded (flow value) or fallen short of (pressure value), to identify it as an intention to speak. If the intention to speak is identified, the speaking apparatus 40 switches from the standby mode into a speaking mode and adjusts the delivery of speaking gas so that the patient can speak. For example, for this purpose, the speaking apparatus 40 adjusts the flow to a value of from about 10 l/min to about 80 l/min, preferably from about 20 l/min to about 60 l/min, more preferably from about 30 l/min to about 50 l/min. If the patient stops speaking, at least the pressure in the speaking tube is increased. The speaking apparatus 40 subsequently switches back into the standby mode. In some embodiments, the flow of speaking gas can also be predetermined on the basis of a (roughly) adjusted speaking loudness. For example, a division into quiet—medium—loud is possible, wherein a lower speaking gas flow is associated with the “quiet” setting than with “medium” and “loud”, and the “loud” setting signifies the maximum speaking gas flow.

The system 1 is configured and designed in such a manner that the system 1 can switch at least temporarily into a speaking mode 63 in which speaking gas is made available to the patient via the speaking tube 22 and enables the patient to speak.

In some embodiments, the speaking apparatus 40 is likewise in the form of a ventilation apparatus.

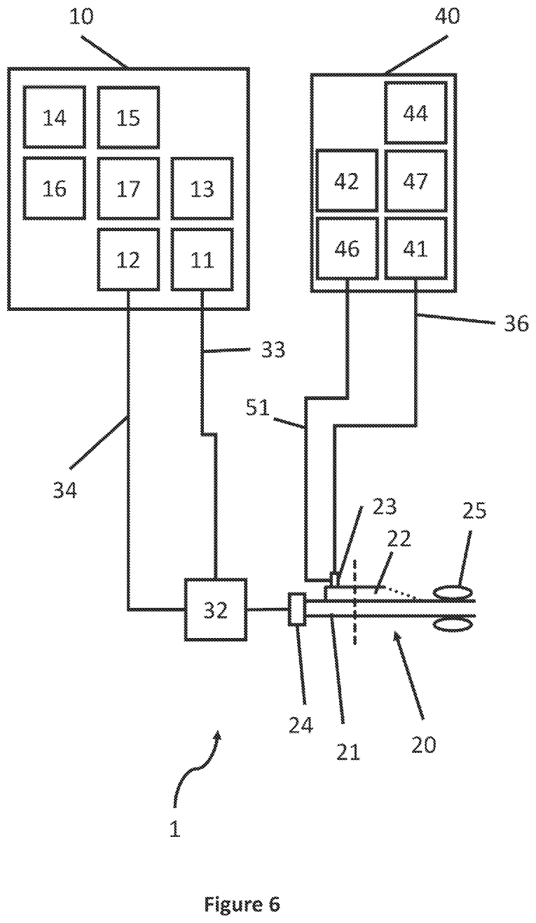

FIG. 6 illustrates an exemplary embodiment of the system 1 in which the speaking apparatus 40 is operated independently of the ventilation apparatus 10. The ventilation apparatus 10 and the speaking apparatus 40 correspond in the essential features to the previously described embodiments. In the embodiment which is shown, the speaking apparatus 40 controls, in particular with reference to the intention of the patient to speak, whether and to what extent speaking gas is provided. In this embodiment, an adaptation to the respiratory phases of the patient is not envisaged, but can additionally also be realized, for example by transmitting information regarding the respiratory phases of the patient from the ventilation apparatus 10 to the speaking apparatus 40.

The intention of the patient to speak is identified, for example, directly via the speaking tube 22. For this purpose, corresponding sensors are arranged in the speaking tube 22 or in the region of the speaking tube 22 and/or of the patient and/or the speaking apparatus 40 and/or the ventilation apparatus 10.

For example, the intention to speak can be identified via an acceleration sensor. This acceleration sensor is arranged by way of example in the speaking tube 22 or in the region of the speaking tube 22 and connected to the speaking apparatus 40 via the sensor connection 51. Control of the deliveries of speaking gas or the supply of speaking gas to the speaking tube 22 is then regulated in accordance with the identification of the intention to speak. When the intention to speak is identified, the speaking apparatus 40 automatically provides a sufficient flow of speaking gas to enable the patient to speak.

Alternatively or additionally, the identification of the intention to speak is also conceivable via a microphone which is arranged in the region of the speaking tube 22 and/or of the patient and/or of the speaking apparatus 40 and/or of the ventilation apparatus 10.

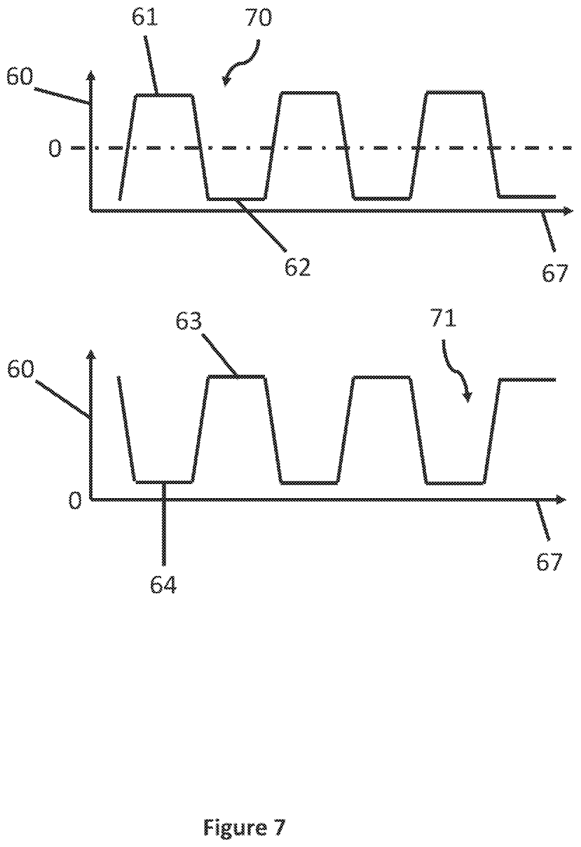

FIG. 7 illustrates by way of example and in greatly simplified form the time curve of the flow 60 of the respiratory gas flow 70, provided by the ventilation apparatus 10, and of the speaking gas flow 71, provided by the ventilation apparatus 10 (exemplary embodiments from FIG. 1 and/or FIG. 2) or by the speaking apparatus 40 (exemplary embodiments from FIGS. 4 to 6). The X axis (time 67) is scaled identically for the two curves and comprises the same period of time. The flow curve 70 of the respiratory gas follows, by way of example, the respiratory phases of the patient. During the inspiration 61, a higher flow is recorded or predetermined than during the expiration 62. By contrast, the curve of the speaking gas flow 71 is the other way around, i.e., the speaking assistance 63 (or else “speaking mode”) takes place during the expiration 62 of the patient, that is to say an increased flow 60 of speaking gas is provided. In the manner of operation illustrated in FIG. 7, the speaking gas flow 71 is not reduced completely to 0 (zero) during the inspiration 62 of the patient, and a small speaking gas flow (and/or pressure) is also maintained during the inspiration 62 of the patient, for example as standby mode 64. In the manner of operation shown in FIG. 7, the speaking assistance 63 periodically changes with the standby mode 64 to the same degree as the respiratory gas flow 70 changes between expiration 61 and inspiration 62.

The speaking gas flow 71 is depicted in such a manner that a positive value describes a speaking gas flow from the gas source to the patient. The higher the value, the higher the flow which arrives at the patient or in the speaking tube.

The system 1 is configured in particular to predetermine a rectangular profile and/or a decreasing flow profile for the speaking gas flow 71. In addition, provision can also be made for the flow profile of the speaking gas flow 71 to have a ramp at the beginning and/or at the end of the delivery of speaking gas. In some embodiments, the flow profile of the speaking gas flow can be freely configured. It should be pointed out that the flow profile of the speaking gas flow 71 preferably does not correspond to the flow profile of the respiratory gas flow of the patient during the expiration 62.

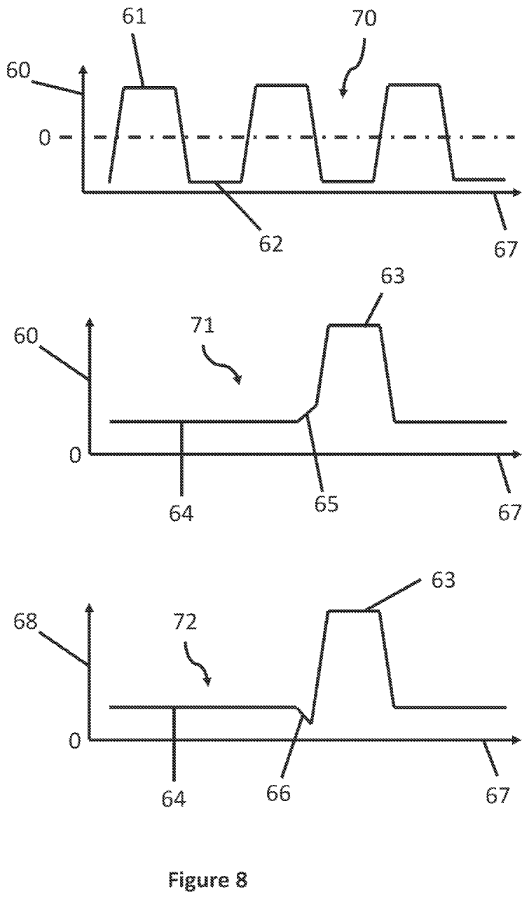

FIG. 8 shows, in greatly simplified form, exemplary flow or pressure curves of the speaking gas or the respiratory gas. The respiratory gas flow 70 follows the respiratory phases of the patient: during the inspiration 61 of the patient a positive flow 60 through the ventilation apparatus 10 is predetermined or recorded, whereas the expiration 62 predetermines or records a negative flow, with a positive flow depicting a flow of respiratory gas to the patient/into the lungs and a negative flow depicting a flow of respiratory gas from the patient/out of the lungs. For example, the delivery of speaking gas is regulated in such a manner that basically no speaking assistance 63 is permitted during the inspiration 61 of the patient.

For example, a small speaking gas flow 71 or speaking gas pressure 72 is predetermined continuously in a standby mode until an intention by the patient to speak is established. For example, an intention of the patient to speak can be established by a flow increase 65 in the speaking gas flow 71 being recorded. If an intention to speak is established, a switch is made from the standby mode to the speaking mode or the speaking assistance 63, which, for example, has the result of increasing the speaking gas flow 71 and enabling the patient to speak.

Alternatively or additionally thereto, identification of the intention to speak by way of a drop in pressure 66 of the speaking gas pressure 72 is also conceivable. Similarly, as with the speaking gas flow 71, the speaking gas pressure 72 is increased when an intention of the patient to speak is identified, by switching over from the standby mode 64 to the speaking assistance 63, in order to enable the patient to speak. The switching over from speaking assistance 63 back into the standby mode 64 can take place, for example, on the basis of a slight increase in pressure (not illustrated) in the speaking tube 22. A slight increase in pressure can arise by the attempt of the patient to close the glottis. Alternatively or additionally, a drop in the speaking gas flow (not illustrated) during the speaking assistance 63 can also lead to a switch into the standby mode 64.

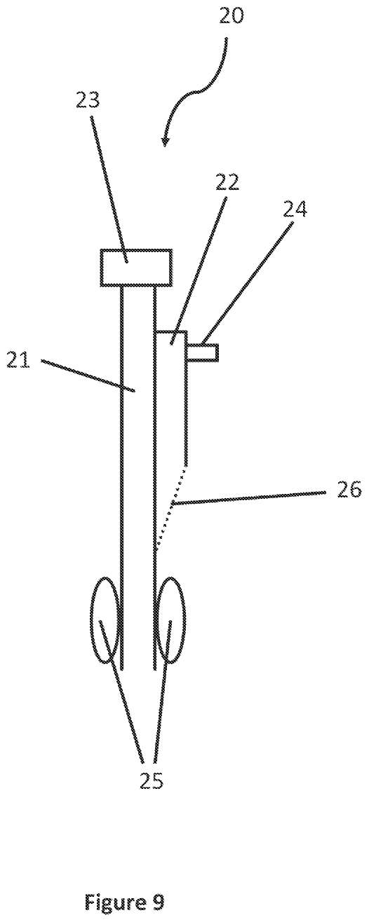

An exemplary embodiment of the patient interface 20 is illustrated schematically in FIG. 9. The patient interface 20 comprises a respiratory tube 21 with a respiratory attachment 23 for attachment to a respiratory gas line 33, for example via a Y piece 32. Furthermore, the patient interface 20 comprises a speaking tube 22 which is at least temporarily supplied with speaking gas via the speaking attachment 24 to a speaking gas line 36, for example coming from a ventilation apparatus 10 and/or a speaking apparatus 40. The speaking tube 22 has a speaking opening 26 through which the speaking gas can escape.

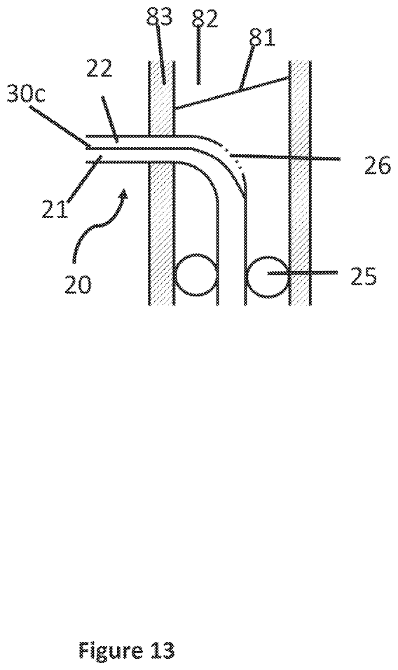

The patient interface 20 is configured and designed in such a manner that the respiratory tube 21 and the speaking tube 22 can be at least partially introduced into the trachea 83 (see FIG. 13) of a patient. The patient interface 20 by way of example is a tracheal cannula, in particular in the form of blocked embodiments. A sealing element 25, for example a cuff or a balloon, is arranged in sections at least around the respiratory tube 21 and seals the patient's trachea around the respiratory tube 21. The respiratory tube 21 is configured to conduct respiratory gas to and/or from the patient's lungs. For example, during the inspiration of the patient, respiratory gas is conducted through the respiratory tube 21 to the patient's lungs and, during the expiration, respiratory gas is conducted away from the patient's lungs. The sealing element 25 is arranged, for example, in such a manner that respiratory gas is substantially, i.e., with the exception of unavoidable leakages, conducted only through the respiratory tube 21 to and away from the patient's lungs.

The speaking opening 26 in the speaking tube 22 is arranged in such a manner that the respiratory gas can escape above the sealing element 25, i.e., is not conducted into the patient's lungs. The speaking opening 26 is also arranged in such a manner that it is positioned between the patient's glottis and the sealing element 25. When the patient's glottis is open, speaking gas can therefore flow out of the speaking tube 22 through the glottis into the patient's oral cavity.

In some embodiments of the patient interface 20, respiratory tube 21 and speaking tube 22 run parallel at least in sections.

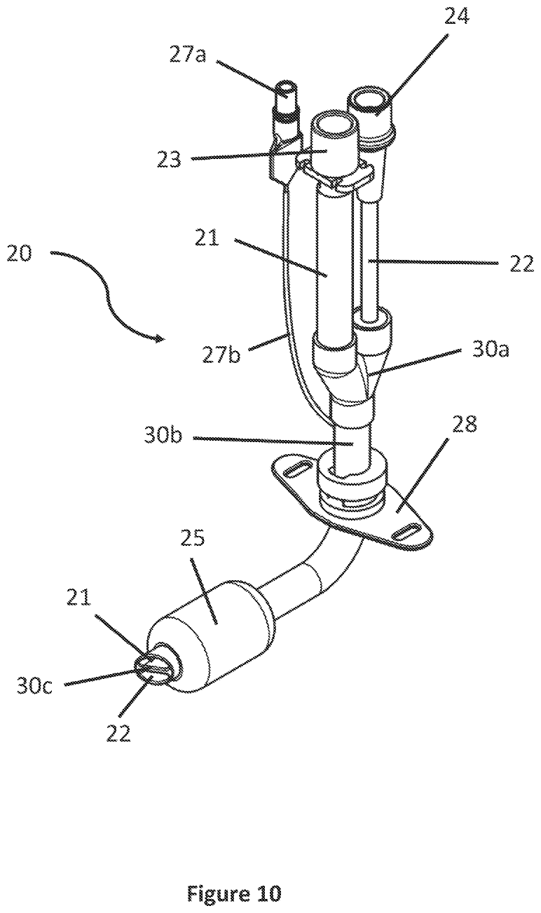

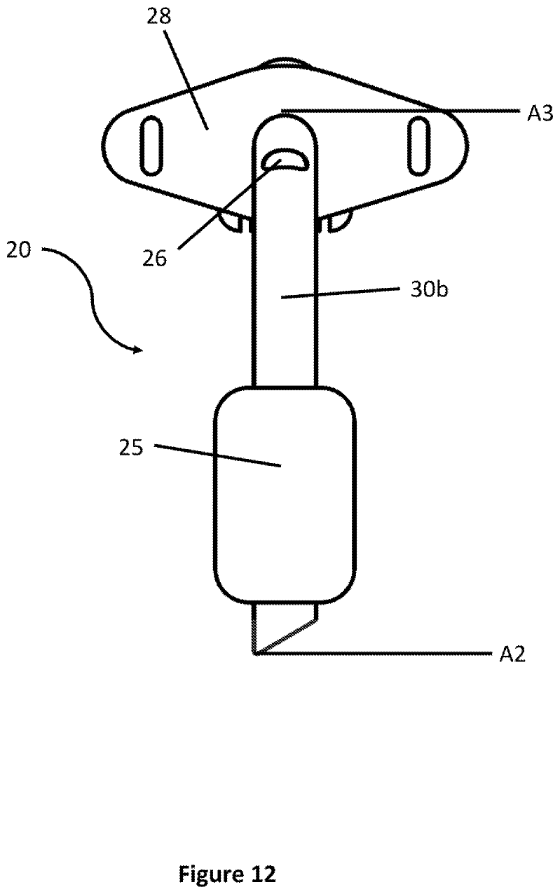

An exemplary embodiment of the patient interface 20 as a tracheal cannula is illustrated in FIGS. 10 to 12. The tracheal cannula is configured in such a manner that it can be introduced through, for example, a trachea section below the glottis into the patient's trachea. In addition to the respiratory tube 21 and the speaking tube 22, the exemplary patient interface also comprises a balloon attachment 27a with a balloon line 27b in order to expand the sealing element 25, which by way of example is in the form of a balloon, and/or to monitor and optionally to adjust (increase/reduce) the pressure in the sealing element 25.

The respiratory tube 21 and the speaking tube 22 are guided in parallel, beginning at the attachments 23, 24, and are guided in a Y piece 30a into a common tube 30b, wherein the respiratory tube 21 and the speaking tube 22 are separated from each other in the tube 30b by a tube wall 30c such that gas cannot pass from the respiratory tube 21 into the speaking tube 22 or vice versa. The tube 30b is, for example, a tube with two lumens, wherein the one lumen serves for the speaking tube 22 and the other lumen serves for the respiratory tube 21. An arrangement of the attachments 23, 24 directly on the Y piece 30a and/or an at least partial integration of the attachments 23, 24 in the Y piece 30a is also possible. In some embodiments, the holding plate 28 is arranged directly on the Y piece 30a.

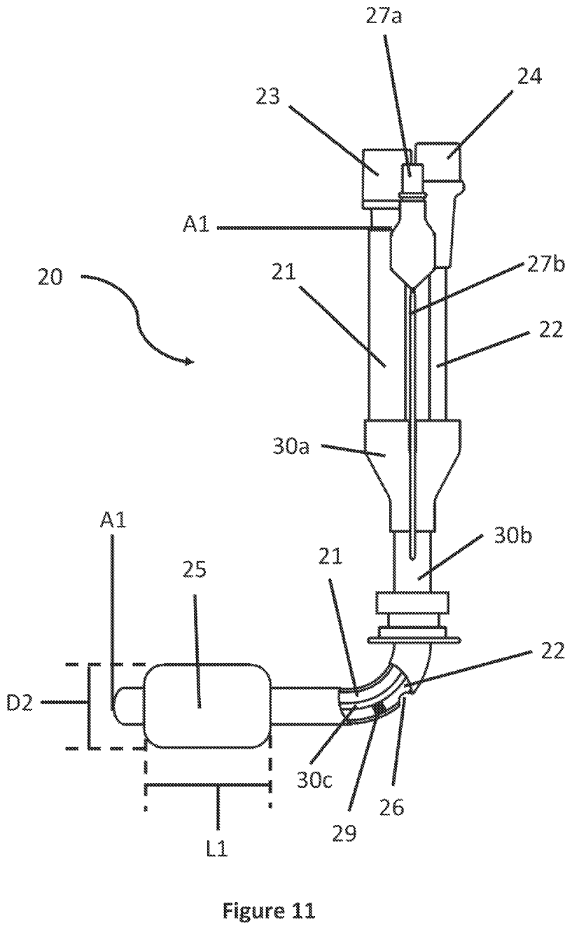

As illustrated in FIG. 11, the speaking tube 22 is blocked downstream of the speaking opening 26 by a tube closure 29 such that no speaking gas passes behind the sealing element 25 and into the patient's lungs. The tube 30b has, for example, a kink/bend, for example at an angle of 90°. The speaking opening 26 is formed, for example, in the kink/bend. Speaking gas can be conducted through the speaking opening 26 out of the speaking tube 22 into the patient's trachea such that the speaking gas can escape through an open glottis into the patient's oral cavity. The sealing element 25 is arranged, for example, in such a manner that it runs around the tube 30b.

The patient interface 20 which is illustrated by way of example in FIGS. 10 to 12 furthermore comprises a holding plate 28 for fixing the patient interface 20 to the patient's neck. For this purpose, the holding plate 28 can be connected, for example, to a band which can be guided around the patient's neck and can hold the patient interface 20 in position.

Over the course of the patient interface (20), for example, the start can be considered to be in the attachments for speaking gas (speaking attachment 24) and respiratory gas (respiratory attachment 23). In the exemplary embodiment, the Y piece 30a which guides the respiratory tube 21 and the speaking tube 22 together into a tube 30b is arranged subsequently, with the speaking tube 22 being separated from the respiratory tube 21 by a tube wall 30c. The tube 30b can therefore be understood as being the tube with two lumens, wherein one lumen conducts the speaking gas and one lumen the respiratory gas. If the patient interface 20, as illustrated, is a tracheal cannula, the tube 30b kinks after the holding plate 28 at an angle of between 45° and 135°. The speaking opening 26 via which the speaking gas can escape from the speaking tube 22 is arranged in this kink/bend. Over the further course the speaking tube 22, after the speaking opening 26, is closed by a tube closure 29, as illustrated in FIG. 11. Before the end of the tube 30b, the sealing element 25 is located on the outer side. The respiratory tube 21 is open toward the end such that respiratory gas can be conducted through the respiratory tube 21 to/from the patient's lungs.

In some embodiments, the tube 30b has a 180° turn, with the holding plate 28 being arranged centrally in the turn. Alternatively, the tube has a kink at an angle of 45° to 90° upstream of the holding plate 28 and also a kink at an angle of 45° to 90° downstream of the holding plate 28. The section of the tube 30b that is guided outside the patient's neck and the section which is guided inside the patient's neck thus run parallel at least in sections.

The patient interface 20 at least partially consists by way of example of PVC (polyvinyl chloride), PUR (polyurethane), PE (polyethylene), EVA (ethylene-vinyl acetate copolymer) and/or ABS (acrylonitrile-butadiene-styrene copolymer). The tube 30c is manufactured by way of example from PVC.

The diameter of the tube 30b is between 10 mm and 20 mm, by way of example from about 11 mm to about 15 mm. The diameter D2 of the sealing element 25, by way of example in the form of an approximately cylindrical cuff, is from about 20 mm to about 40 mm, by way of example from about 24 mm to about 30 mm and/or from about 26 mm to about 28 mm. The length L1 of the sealing element is from about 30 mm to about 50 mm, by way of example from about 35 mm to about 45 mm. The overall length in an extended state from A1, for example indicated by the transition from the respiratory attachment 23 to the respiratory tube 21, as far as A2, which constitutes the end of the tube 30b, is from about 100 mm to about 250 mm, for example from about 150 mm to about 200 mm. The length of the section of the tube upstream of the holding plate 28, between A2 and A3, for example in a curved state, is from about 50 mm to about 110 mm, and in some embodiments from about 70 mm to about 100 mm. The end A1 of the tube 30b is, for example, beveled.

The patient interface 20 in FIG. 13 is by way of example a tracheal cannula which is introduced below the patient's glottis 81, for example through a trachea incision, into the patient's trachea 82. The sealing element 25 is arranged in such a manner that the trachea 82 seals at least around the respiratory tube 21 below the patient's glottis 81. The speaking opening 26 of the speaking tube 22 is arranged in such a manner that speaking gas is conducted out of the speaking tube 22 below the glottis 81 but above the sealing element 25 into the patient's trachea. If the patient opens the glottis 81, the speaking gas can flow into the patient's oral cavity and enables the patient to speak.

To sum up, the present invention provides:

1. A system for assisting a patient in speaking, wherein the system comprises at least one ventilation apparatus and a patient interface, the ventilation apparatus comprising at least one controllable respiratory gas source and being configured to identify two or more respiratory phases, at least inspiration and expiration, of the patient, and the patient interface comprising at least a speaking tube and a respiratory tube and being configured to conduct speaking gas to the patient via the speaking tube and to conduct respiratory gas to and/or from the patient via the respiratory tube, the system being configured to provide speaking gas to the patient at least temporarily in a speaking mode in order to enable speaking.

2. The system of item 1, wherein the patient interface is a tracheal cannula.

3. The system of at least one of the preceding items, wherein the system is configured to switch into the speaking mode only during the expiration of the patient.

4. The system of at least one of the preceding items, wherein the system is configured and designed to switch into a standby mode while the speaking mode is not active, and wherein, in the standby mode, a lower pressure and/or smaller flow of speaking gas is present in the speaking tube than a pressure and/or flow in the speaking mode.

5. The system of at least one of the preceding items, wherein the system is configured and designed so as, during a standby mode, to predetermine a pressure and/or flow of speaking gas, at which the patient can close a glottis or can keep same closed.

6. The system of at least one of the preceding items, wherein the system is configured such that a speaking function can be activated and deactivated, with switching into the speaking mode being made possible only when the speaking function is activated.

7. The system of at least one of the preceding items, wherein the system is configured such that, when the speaking function is deactivated, no speaking gas is delivered to the speaking tube.

8. The system of at least one of the preceding items, wherein the system is configured to switch over periodically between a standby mode and the speaking mode, the system switching into the standby mode during the inspiration of the patient and switching into the speaking mode during the expiration of the patient.

9. The system of at least one of the preceding items, wherein the system is configured and designed to identify an intention of the patient to speak and switches into the speaking mode only when an intention of the patient to speak is identified.

10. The system of item 9, wherein the system is configured and designed to identify the intention of the patient to speak on the basis of a pressure drop and/or increase in flow of the speaking gas in the speaking tube.

11. The system of item 9, wherein the system is configured and designed to identify the intention of the patient to speak via at least one acceleration sensor and/or at least one microphone.

12. The system of at least one of the preceding items, wherein the respiratory gas source is configured and designed to deliver speaking gas and respiratory gas.

13. The system of at least one of the preceding items, wherein the system further comprises a speaking controller and at least one control valve, the speaking controller being configured to control the control valve and the control valve being connected in a gas-conducting manner at least to the speaking tube of the patient interface and being connected to a Y piece connected at least to the respiratory tube of the patient interface and both respiratory gas and speaking gas being conducted through the control valve.

14. The system of item 13, wherein the system is configured to switch the control valve in such a manner that, at least in the speaking mode, speaking gas is delivered to the speaking tube.

15. The system of at least one of items 13 and 14, wherein the control valve is switchable in such a manner that, during a standby mode, a pressure and/or flow of speaking gas is maintained in the speaking tube, the pressure and/or flow being lower/smaller than a pressure and/or flow of the speaking gas in the speaking mode.

16. The system of at least one of the preceding items, wherein, in addition to the ventilation apparatus, the system comprises at least one speaking apparatus comprising at least one gas source which is configured to at least temporarily deliver speaking gas.

17. The system of item 16, wherein the speaking apparatus is configured to switch over between the speaking mode and a standby mode.

18. The system of at least one of items 16 and 17, wherein the speaking apparatus is connected to the ventilation apparatus and the speaking apparatus can be coupled to the ventilation apparatus.

19. The system of at least one of items 16 to 18, wherein the speaking apparatus is configured to receive information regarding respiratory phases of the patient from the ventilation apparatus and to control a speaking gas flow on the basis of said information.

20. The system of at least one of items 16 to 19, wherein the speaking apparatus is configured to identify respiratory phases of the patient, at least the inspiration and expiration, and to switch into the speaking mode or a standby mode on the basis of the respiratory phases.

21. The system of at least one of items 16 to 20, wherein the speaking apparatus is configured and designed to identify an intention of the patient to speak and, when an intention to speak is identified, to switch into the speaking mode.

22. A method for controlling a delivery of speaking gas, wherein the method comprises predetermining a speaking gas flow and/or speaking gas pressure in a speaking mode to a first value and, in a standby mode, setting it to a second value, the first value being higher than the second value and the first value being set in such a manner that it is made possible for a user to speak.

23. The method of item 22, wherein a switch is periodically made between the speaking mode and the standby mode.

24. The method of at least one of items 22 and 23, wherein switching over from the standby mode into the speaking mode presupposes that an intention to speak has been identified, wherein the intention to speak is identified by a drop in a speaking gas pressure during the standby mode and/or an increase in a speaking gas flow during the standby mode.

25. The method as claimed in at least one of items 22 to 24, wherein delivery of speaking gas takes place in a rectangular profile and/or a decreasing flow profile, with the delivery of speaking gas taking place independently of an exhalation profile of the patient.

26. A patient interface for at least partially introducing into the trachea of a patient, wherein the interface comprises at least one respiratory tube and at least one speaking tube and at least one sealing element, a gas being able to be conducted through the respiratory tube and the speaking tube independently of each other, the speaking tube being connected via a speaking attachment to a speaking gas line and being at least temporarily supplied with speaking gas, and the respiratory tube being connected via a respiratory attachment with a Y piece to at least one respiratory gas line and being at least temporarily supplied with respiratory gas, and the speaking tube comprising a speaking opening which is arranged between the speaking attachment and the sealing element.

### LIST OF REFERENCE SIGNS

1 System
10 Ventilation apparatus
11 Respiratory gas source
12 Exhaled air device
13 Speaking controller
14 Control unit
15 Evaluation unit
16 Sensor unit
17 User interface
18 Interface
20 Interface
21 Respiratory tube
22 Speaking tube
23 Respiratory attachment
24 Speaking attachment
25 Sealing element
26 Speaking opening
27a Balloon attachment
27b Balloon line
28 Holding plate
29 Tube closure
30a Y piece
30b Tube
30c Tube wall
31 Valve
32 Y piece
33 Respiratory gas line
34 Respiratory gas line
35 Control line
36 Speaking gas line
37 (3/2-way) valve
38 Control line
39 Outgoing air
40 Speaking apparatus
41 (Speaking) gas source
42 Control unit
43 Interface
44 User interface
45 Valve controller
46 Sensor unit
47 Evaluation unit
51 Sensor connection
60 Flow
61 Inspiration
62 Expiration
63 Speaking assistance
64 Standby
65 Increase in flow
66 Drop in pressure
67 Time
68 Pressure
70 Respiratory gas flow
71 Speaking gas flow
72 Speaking gas pressure
81 Glottis
82 Trachea
83 Neck

What is claimed is:

1. A system for assisting a patient in speaking, wherein the system comprises at least one ventilation apparatus and a patient interface, the ventilation apparatus comprising at least one controllable respiratory gas source and being configured to identify two or more respiratory phases, at least inspiration and expiration, of the patient, and the patient interface comprising at least a speaking tube and a respiratory tube and being configured to conduct speaking gas to the patient via the speaking tube and to conduct respiratory gas to and/or from the patient via the respiratory tube, the system being configured to provide speaking gas to the patient at least temporarily in a speaking mode in order to enable speaking.

2. The system of claim 1, wherein the patient interface is a tracheal cannula.

3. The system of claim 1, wherein the system is configured to switch into the speaking mode only during the expiration of the patient.

4. The system of claim 1, wherein the system is configured and designed to switch into a standby mode while the speaking mode is not active, and wherein, in the standby mode, a lower pressure and/or smaller flow of speaking gas is present in the speaking tube than a pressure and/or flow in the speaking mode.

5. The system of claim 1, wherein the system is configured and designed so as, during a standby mode, to predetermine a pressure and/or flow of speaking gas, at which the patient can close a glottis or can keep same closed.

6. The system of claim 1, wherein the system is configured such that a speaking function can be activated and deactivated, with switching into the speaking mode being made possible only when the speaking function is activated.

7. The system of claim 1, wherein the system is configured such that, when the speaking function is deactivated, no speaking gas is delivered to the speaking tube.

8. The system of claim 1, wherein the system is configured to switch over periodically between a standby mode and the speaking mode, the system switching into the standby mode during the inspiration of the patient and switching into the speaking mode during the expiration of the patient.

9. The system of claim 1, wherein the system is configured and designed to identify an intention of the patient to speak and switches into the speaking mode only when an intention of the patient to speak is identified.

10. The system of claim 9, wherein the system is configured and designed to identify the intention of the patient to speak on the basis of a pressure drop and/or increase in flow of the speaking gas in the speaking tube.

11. The system of claim 9, wherein the system is configured and designed to identify the intention of the patient to speak via at least one acceleration sensor and/or at least one microphone.

12. The system of claim 1, wherein the respiratory gas source is configured and designed to deliver speaking gas and respiratory gas.

13. The system of claim 1, wherein the system further comprises a speaking controller and at least one control valve, the speaking controller being configured to control the control valve and the control valve being connected in a gas-conducting manner at least to the speaking tube of the patient interface and being connected to a Y piece connected at least to the respiratory tube of the patient interface and both respiratory gas and speaking gas being conducted through the control valve.

14. The system of claim 13, wherein the system is configured to switch the control valve in such a manner that, at least in the speaking mode, speaking gas is delivered to the speaking tube.

15. The system of claim 13, wherein the control valve is switchable in such a manner that, during a standby mode, a pressure and/or flow of speaking gas is maintained in the speaking tube, the pressure and/or flow being lower/smaller than a pressure and/or flow of the speaking gas in the speaking mode.

16. The system of claim 1, wherein, in addition to the ventilation apparatus, the system comprises at least one speaking apparatus comprising at least one gas source which is configured to at least temporarily deliver speaking gas.

17. The system of claim 16, wherein the speaking apparatus is configured to switch over between the speaking mode and a standby mode.

18. The system of claim 16, wherein the speaking apparatus is connected to the ventilation apparatus and the speaking apparatus can be coupled to the ventilation apparatus.

19. A method for controlling a delivery of speaking gas, wherein the method comprises predetermining a speaking gas flow and/or speaking gas pressure in a speaking mode to a first value and, in a standby mode, setting it to a second value, the first value being higher than the second value and the first value being set in such a manner that it is made possible for a user to speak.

20. A patient interface for at least partially introducing into the trachea of a patient, wherein the interface comprises at least one respiratory tube and at least one speaking tube and at least one sealing element, a gas being able to be conducted through the respiratory tube and the speaking tube independently of each other, the speaking tube being connected via a speaking attachment to a speaking gas line and being at least temporarily supplied with speaking gas, and the respiratory tube being connected via a respiratory attachment with a Y piece to at least one respiratory gas line and being at least temporarily supplied with respiratory gas, and the speaking tube comprising a speaking opening which is arranged between the speaking attachment and the sealing element.
